(19) United States
(12) Patent Application Publication
(10) Pub. No.: US 2005/0019937 A1
(43) Pub. Date: Jan. 27, 2005

(54) Assay and kit for homocysteine

(75) Inventors: Chia-Chann Shiue, Taoyuan (TW); Su-Jan Lee, Taipei (TW); Tzu-I Wu, Changhua (TW); Mei-Fang Su, Hsinchu (TW); Jen-Hao Chang, Changhua (TW); Chih-Chung Wu, Hsinchu (TW)

Correspondence Address: SUGHRUE MION, PLLC, 2100 PENNSYLVANIA AVENUE, N.W., SUITE 800, WASHINGTON, DC, 20037, US

(73) Assignee: INDUSTRIAL TECHNOLOGY RESEARCH INSTITUTE

(21) Appl. No.: 10/621,371

(22) Filed: Jul. 18, 2003

Related U.S. Application Data
(63) Continuation-in-part of application No. 10/412,303, filed on Apr. 14, 2003, now abandoned.

(30) Foreign Application Priority Data
Nov. 29, 2002 (TW) 91134803

(51) Int. Cl. G01N 33/00

(52) U.S. Cl. 436/86

(57) ABSTRACT

A method and kit is provided for assaying homocysteine in a biological sample containing homocysteine and cysteine. A competing compound with an amino group (—NH₂) is mixed with the biological sample. An aldehyde, e.g. o-phthalaldehyde, is added to the biological sample to form homocysteine complex with fluorescence. The concentration of homocysteine in the biological sample is determined according to the fluorescent intensity.

### BACKGROUND OF THE INVENTION

1. Field of the Invention

The invention relates to a chemical assay and in particular to an assay and kit for Homocysteine detection and detection in bio-samples.

2. Description of the Related Art

Homocysteine (Hcy), an thiol-containing amino acid, is a metabolic intermediate of both methionine (Met) and cysteine (Cys) production and is found normally in the body. Various studies have found that persons with elevated levels of homocysteine in their blood are at an increased risk of heart and vessel disease. McCully et al. first reported the association of blood plasma homocysteine levels with risk from cardiovascular disease (McCully et al. Am. J. Pathol. (1969) 56: 111-128.). Some studies also indicate that the homocysteine may make blood more likely to clot by increasing the stickiness of blood platelets and clots can block blood flow, causing a heart attack or stroke. In addition, homocysteine is viewed as the first risk factor for atherosclerosis, believed to exert its effects through a mechanism involving oxidative damage.

Homocysteine metabolism is linked to that of several vitamins, especially folic acid, and B12. It is observed that deficiencies of those vitamins may cause elevated levels of homocysteine. Since January 1998, the US Food and Drug Administration has required that all enriched grain products contain 140 μg of folic acid per 100 g, a level considered to decrease homocysteine levels.

Homocysteine and cysteine are sulfhydryl amino acids both of which comprise three functional groups (—NH₂, —COOH and —SH) in one molecule. There is only a single carbon difference between the two compounds.

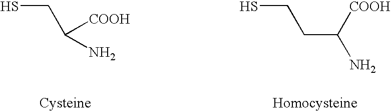

Similarity among functional groups is why it is very difficult to distinguish between homocysteine and cysteine. The exploited methods for the assay of homocysteine have been gas chromatography-mass spectrometry (GC-MS), high-performance liquid chromatography (HPLC), high-performance capillary electrophoresis (HPCE), fluorescence polarization immunoassay (FPIA) and enzyme immunoassay (EIA). Unfortunately, chromatographic methods have the disadvantages of being slow and labor intensive. It is problematic to measure such low concentration homocysteine and its disulphides in human plasma. As well as low concentration, homocysteine is also susceptible to oxidation, resulting in difficulty in detection of homocysteine in human plasma.

Edwin discloses a non-immunoassay assay for homocysteine (U.S. Pat. No. 6,265,220). Cysteine and homocysteine react with a compound that comprises a cis-1,4-dioxo-2-butene moiety or hydrolytically derived precursor thereto with different behavior. In this patent, the maximum emission wavelength of cysteine-adduct is 566 nm and the maximum emission wavelength of homocysteine-adduct is 577 nm. Because cysteine-adduct and homocysteine-adduct have similar fluorescence, they are difficult to distinguish from each other by this assay. In addition, antibody labeled with a quencher is added to remove the interference between the adducts of cysteine and o-phthalaldehyde, because homocysteine and cysteine had the similar maximum emission wavelength at 520 nm when HEPES buffer system was used. Because the emission fluorescence was broadband, the fluorescence signals of cysteine and homocysteine were difficult to distinguish.

Reuel et al. disclose an immunoassay for homocysteine (U.S. Pat. No. 5,478,729). Tris(2-carboxyethyl)phosphine was added to reduce disulfides in plasma and then p-bromoacetylbenzoic acid was added to react with cysteine and homocysteine. The methods of this invention involve chemically modifying both the analyte and the homolog to increase their immunogenicity and facilitate antibody recognition. The reagent containing sulfhydryl groups reacts with homocysteine not to form a ring and with cysteine to form a 6-membered ring. HPLC is applied to quantify homocysteine. The other method uses antibodies capable of specifically binding to the modified homocysteine after the ring comes into existence. The modified cysteine-adduct does not form an immunecomplex, and cysteine will not interfere with the detection of homocysteine. The disadvantage of this patent is a complicated treatment of sample for clinic applicability was disclosed.

Currently, the methods for homocysteine assay are both time-consuming and expensive. Frantzen et al. (Frantzen et al. Clin. Chem. (1998) 44 : 311-316.) convert the homocysteine to S-adenosyl homocysteine and detect S-adenosyl homocysteine using monoclonal antibodies in an enzyme-immunosorbent assay (EIA). Although it is an automatic procedure, it takes 2.5 hours for one test.

Dias et al. treated the reduced plasma with a derivatizing agent such as 4-aminosulfonyl-7-fluoro-2,1,3-benzoxydiazole to form a fluorescent adduct with cysteine and homocysteine (Dias et al. Clin. Chem. (1998) 44 : 2199-2201.). The adducts of cysteine and homocysteine are separated by HPLC. However, a single detection requires one hour. It is a time-consuming assay for homocysteine detection. This procedure is available as a kit from BioRad at a cost of $7˜7.5 per assay.

Yuying et al. disclose a high specificity homocysteine assay for biological samples (U.S. Pat. No. 5,985,540). Yuying et al. provide a detection assay of homocysteine in biological samples without interference from cysteine or methionine that are routinely present in such samples, because a particular homocysteinase enzyme “γ-glutamylcysteine synthetase” was used to limit any interference from cysteine. Hydrogen disulfide is produced when cysteine and homocysteine contacts enzyme and the amount of hydrogen disulfide is equal to the amount of homocysteine. This method still presents a time-consuming and complex assay.

Rozella et al. disclose a method for measuring the amount of L-homocysteine and/or L-methionine in a solution (U.S. Pat. No. 5,885,767). The 2-ketobutyrate is produced when L-homocysteine and/or L-methionine contacts methionine gamma-lyase, so the amount of L-homocysteine and/or L-methionine are based on the amount of 2-ketobutyrate formed. The advantage of this method is that L-homocysteine and L-methionine produce similar responses, because methionine is routinely present in biological samples.

Erling et al. disclose an enzymetic assay for homocysteine and a kit therefor (U.S. Pat. No. 5,631,127). S-adenosyl-homocysteine hydrolase (SAH-hydrolase) is used to catalyze the reaction of homocysteine and adenosine (equilibrium constant=10⁶ M⁻¹). The disadvantage is that the equilibrium constant is only 10⁶ M⁻¹ for the catalytic reaction of S-adenosyl-homocysteine hydrolase. This causes a critical error when the concentration of homocysteine is a very low initial concentration.

Candra et al. disclose methods for detecting homocysteine, comprising converting homocysteine in the sample to homocysteine thiolactone, reacting free thiol-containing compounds in the sample with a thiol-capturing agent, reconverting homocysteine thiolactone to homocysteine, and detecting the presence of homocysteine in the sample (U.S. Pat. No. 6,020,206). In the method, reconverting homocysteine thiolactone to homocysteine is a complicated treatment, because the ring opening reaction is investigated at 80° C. for 30 minutes.

Therefore, there is a need for a fast and economical assay to determine Homocysteine in bio-samples excluding cysteine interference.

### SUMMARY OF THE INVENTION

One object of the present invention is to provide a kit and assay for rapid detection of homocysteine in a bio-sample that may contain cysteine as well.

Another object of the invention is to provide an economical kit and assay for clinical detection of homocysteine in a bio-sample that also contains cysteine.

Still another object of the invention is to provide a kit and assay for accurate detection of low concentration homocysteine in a bio-sample that also contains cysteine.

Accordingly, the present invention provides a kit for determining homocysteine in a bio-sample containing homocysteine and cysteine. The kit includes a competing agent that comprises at least one compound with an amino group to compete with cysteine in the sample, and a reactive agent that forms a fluorescent complex with homocysteine but does not form fluorescent complex with the competing agent.

Accordingly, the present invention further provides a method for determining homocysteine in a bio-sample, comprising adding a competing agent with an amino group to the bio-sample as mixture (a). An aldehyde compound is then added to mixture (a) to form a fluorescent complex as mixture (b). The fluorescent intensity of mixture (b) is detected to determine the amount of homocysteine present in the bio-sample.

Accordingly, the present invention further provides a method for determining homocysteine in the presence of cysteine in a bio-sample, comprising the following steps. First, a competing agent containing tris-(hydroxymethyl)aminomethane (TRIS) is added to the bio-sample as mixture (a). Second, o-phthalaldehyde solution is added to mixture (a) to form fluorescent complex mixture (b). Finally, the fluorescent intensity of mixture (b) is detected to determine the amount of homocysteine in the bio-sample.

### DESCRIPTION OF THE DRAWINGS

For a better understanding of the present invention, reference is made to a detailed description to be read in conjunction with the accompanying drawings, in which:

FIG. 1 is a spectrum showing the absorption of homocysteine-phthalaldehyde complex with 525 nm light emission according to one embodiment of the present invention;

FIGS. 2A to 2C show the fluorescent spectra of cysteine and homocysteine reacting with various competing agents according to one embodiment of the present invention;

FIG. 3A shows the fluorescent spectra of homocysteine in the TRIS buffer with various reaction times according to one embodiment of the present invention;

FIG. 3B shows the fluorescent spectra of homocysteine in the TRIS buffer reacting for 1 to 5 minutes according to one embodiment of the present invention;

FIG. 4 shows a calibration curve with standard homocysteine and serum standard addition curve respectively according to one embodiment of the present invention; and

FIG. 5 shows the fluorescent spectra of cysteine and homocysteine with acetamide as the competing agent according to one embodiment of the present invention.

### DETAILED DESCRIPTION OF THE INVENTION

Clinical bio-samples, such as human serum or urine, usually contain cysteine and homocysteine that can interfere with each other during conventional detection because of similar chemical response. The present invention provides a kit and a method thereof to determine homocysteine accurately in bio-samples even in the presence of cysteine.

Chemicals used herein are obtained from Merck (Darmstadt), Aldrich Chemical Company (Milwaukee, Wis.) and Sigma (St. Louis, Mo.). All solutions were prepared in water, except o-phthalaldehyde (OPA), prepared in 20% ethanol or methanol solution.

### EXAMPLE 1

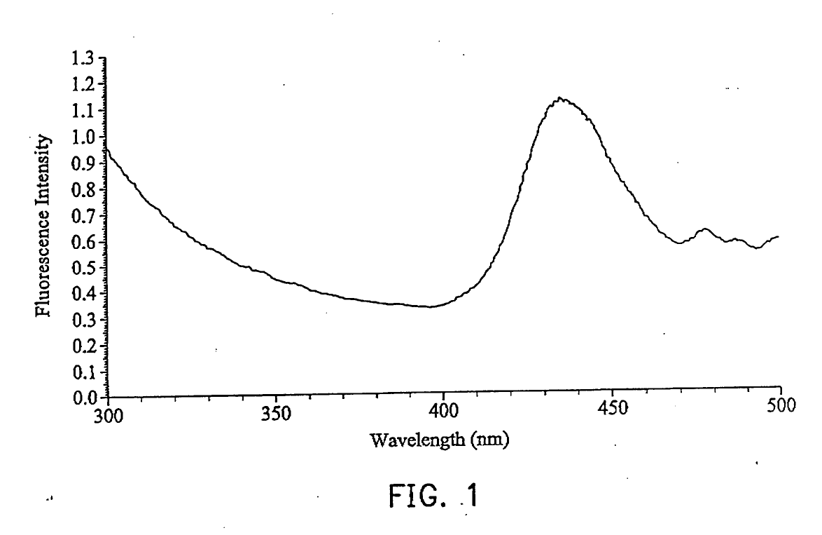

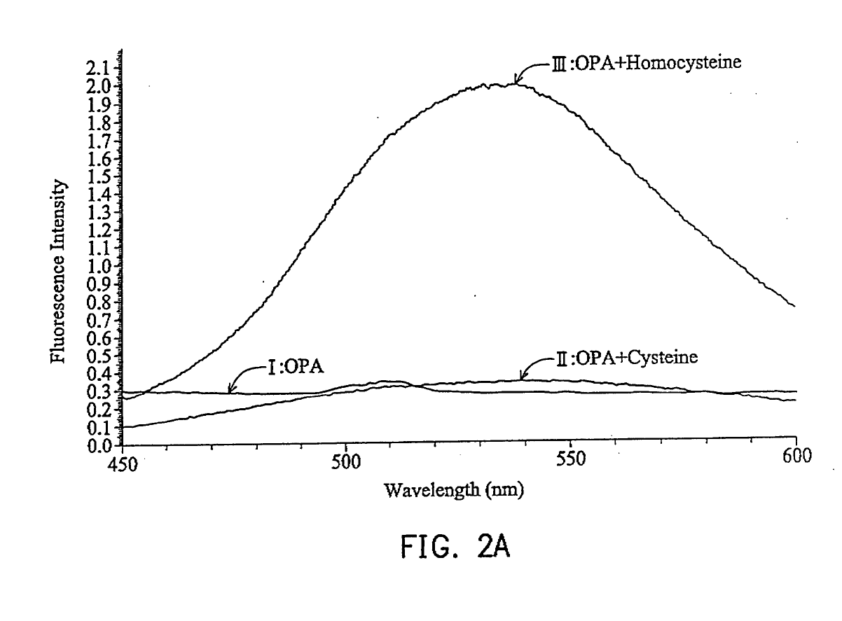

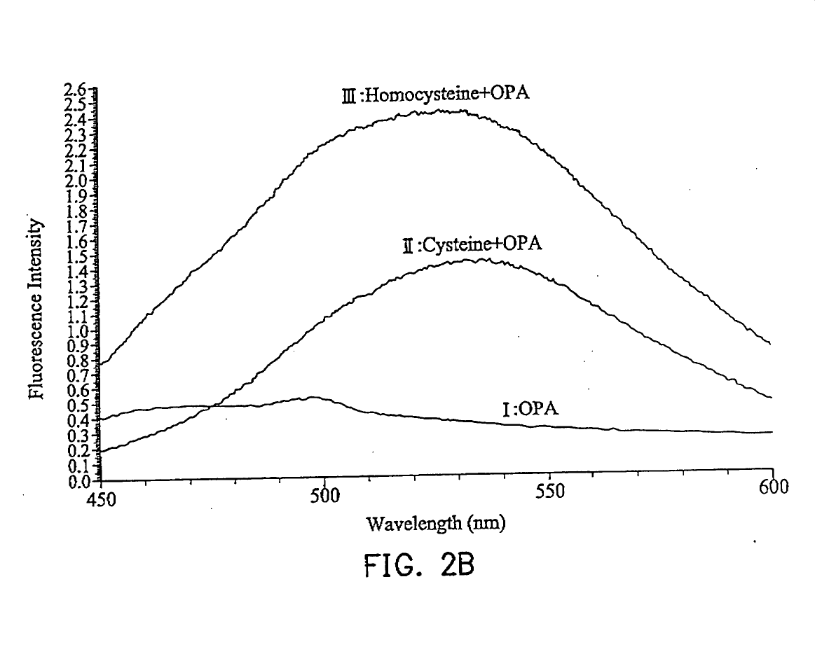

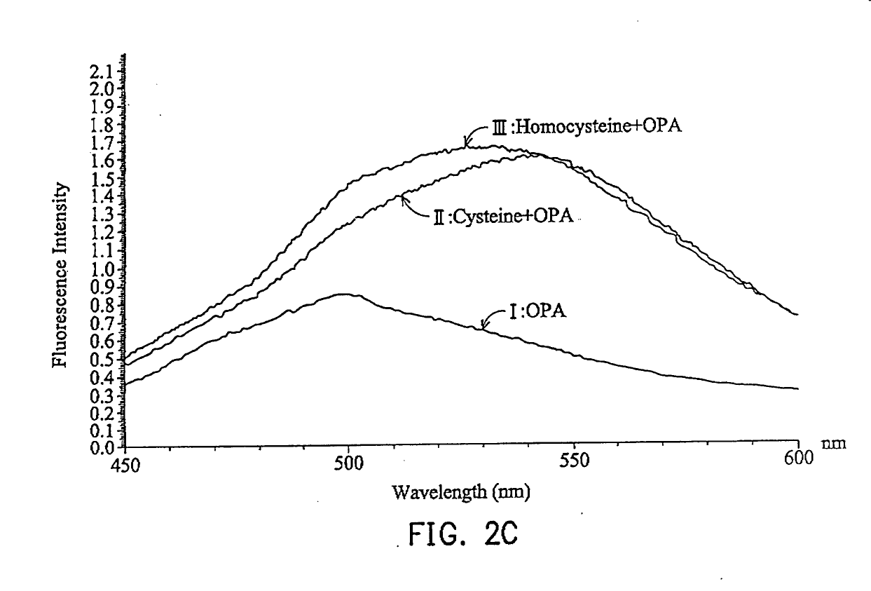

1 ml Tris-(hydroxymethyl)aminomethane (TRIS) buffer (100 mM), containing 1 mM ethylenediamine tetraacetic acid (EDTA) and 18 mM sodium borohydride (NaBH₄) were added to 1 ml solution containing homocysteine, cysteine, and water respectively, stirring for 2 minutes at room temperature. The mixtures were combined with 0.018 mM o-phthalaldehyde (OPA) in 20% aqueous methanol to form fluorescent complex. The emission spectra were detected with 525 nm to monitor the absorption from 300 to 500 nm, as shown in FIG. 1. The fluorescent adduct was monitored with 450˜600 nm (λmax=524 nm) using excitation with 436 nm light. The fluorescence of homocysteine adduct was enhanced after standing 4 min and the fluorescence of cysteine adduct disappeared after standing 4 min, so cysteine and homocysteine in a bio-sample can be separated by way of different spectrometric characteristics, as shown in FIG. 2A.

FIG. 2A shows fluorescent spectra of three samples from wavelength 450˜600 nm at the fourth minute. Curve I is the fluorescence spectrum of the blank solution without homocysteine and cysteine, that is, containing OPA only. Curve II is the cysteine sample that contains cysteine and OPA. Curve III is the homocysteine sample that contains homocysteine and OPA. Since TRIS also comprises an amino group (—NH₂), it competes OPA with the amino group of homocysteine and cysteine. The reaction of OPA and TRIS is as follows:

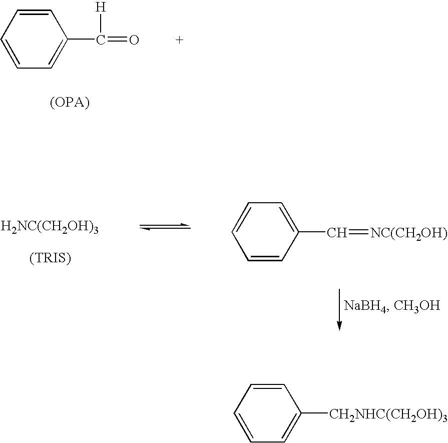

The reactions of OPA and homocysteine/cysteine are as follows:

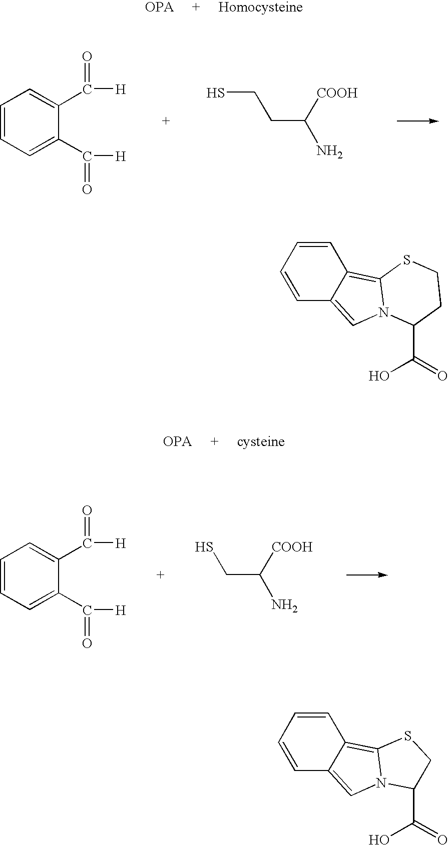

TRIS has an amino group (—NH₂) to react with o-phthalaldehyde, so the TRIS in the buffer will compete for the bond site of o-phthalaldehyde with cysteine and homocysteine.

The product reacted by OPA and TRIS is not excited with fluorescence as curve I in FIG. 2A. Since curve II in FIG. 2A does not show excited fluorescence, TRIS is more reactive than cysteine to react with OPA in the sample. However, curve III in FIG. 2A shows that homocysteine is more reactive than TRIS to react with OPA and form a complex that can be excited to form fluorescence peak.

If the TRIS buffer is substituted with phosphate buffer or N-2-Hydroxyethylpiperazine-2-N′-ethanesulfonic acid (HEPES) buffer, the fluorescence of cysteine complex is similar to homocysteine complex. The results shown in FIG. 2B and FIG. 2C reveal that the fluorescence of cysteine can't be quenched in HEPES buffer and phosphate buffer.

In FIG. 2B, curve I, the blank sample, still shows no fluorescence. However, curve II, the sample containing cysteine, showing high fluorescence and therefore cysteine is more competitive than phosphate in reacting with o-phthalaldehyde. Similarly, curve II, the sample containing homocysteine, also shows high fluorescence. In FIG. 2C, curve I, the blank sample, has shown certain fluorescence by the product of HEPES and o-phthalaldehyde. Both curve II and III, samples containing cysteine and homocysteine respectively, show strong fluorescence by reacting with HEPES. It is obvious that phosphate and HEPES are not superior agents to compete with cysteine to react with o-phthalaldehyde.

In FIGS. 2A to 2C, TRIS buffer is the preferred competing agent among the three buffers. High concentration TRIS is superior to cysteine in reacting with o-phthalaldehyde and forms a non-fluorescent adduct but the reactivity thereof is lower than homocysteine that can form a strong fluorescent complex with o-phthalaldehyde. The competition of TRIS is disadvantageous for cysteine to react with o-phthalaldehyde, because cysteine reacts with o-phthalaldehyde to form a strained 5-membered fused ring that is favorably formed at elevated temperature. On the contrary, homocysteine reacts with o-phthalaldehyde rapidly, because a 6-membered ring with highly fluorescence is formed. TRIS quenches the fluorescence of cysteine but not homocysteine.

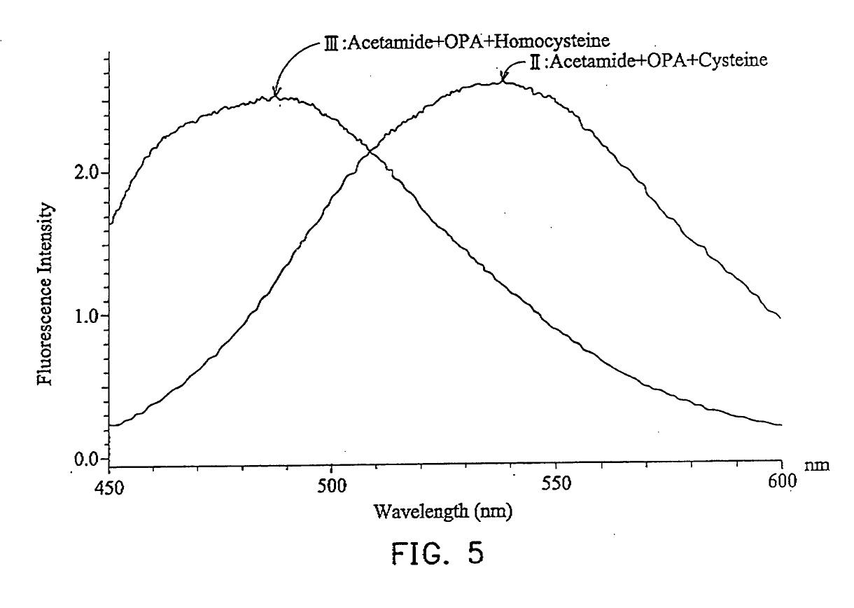

In another embodiment of the present invention, acetamide (CH₃CONH₂) is the competing agent as shown in FIG. 5. 1 ml 100 mM acetamide buffer containing 1 mM ethylenediamine tetraacetic acid (EDTA) and 18 mM sodium borohydride were added to 1 ml sample solution containing homocysteine, cysteine, and water respectively at room temperature. After 2 minutes, the mixtures were combined with 100 mM o-phthalaldehyde in 20% aqueous methanol respectively and fluorescence was measured. In FIG. 5, Curve II shows the fluorescent emission spectrum of cysteine-acetamide complex and curve III is that of homocysteine-acetamide complex. When acetamide is chosen as the competing agent, the fluorescent emission of cysteine-acetamide complex is very low, near wavelength of 450 nm. The concentration of homocysteine can be calculated by detecting the fluorescence of homocysteine-acetamide complex at 450 nm.

### EXAMPLE II

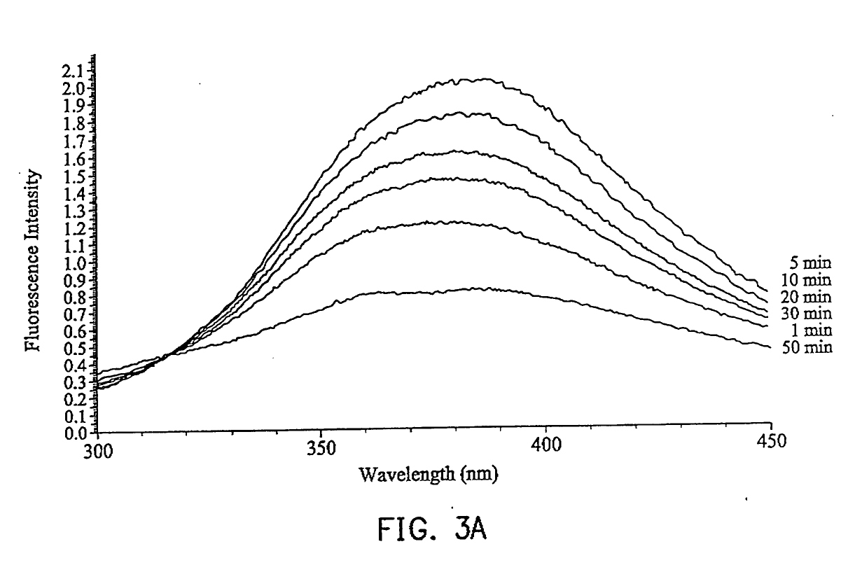

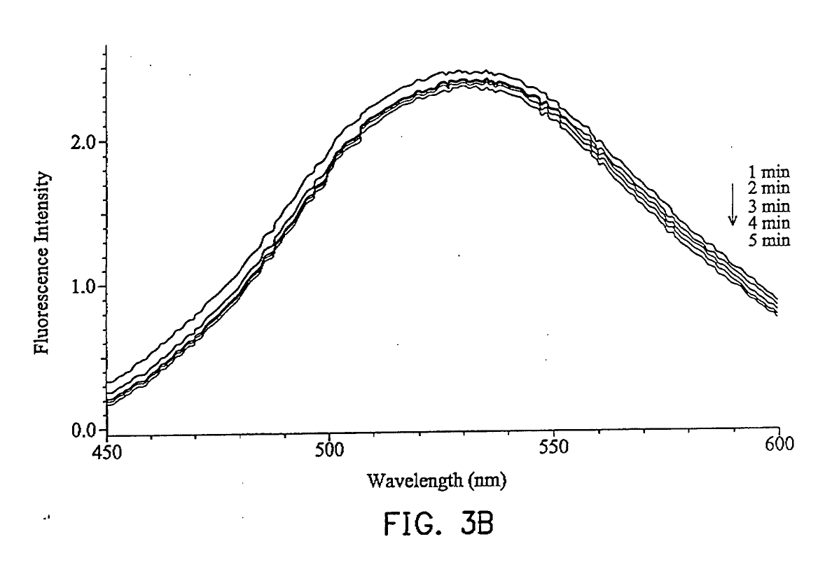

This embodiment of the present invention shows the preferred detection time of the homocysteine assay. 100 mM TRIS buffer containing 1 mM EDTA and 18 mM NaBH₄ was added to samples containing homocysteine with equivalent volume to react for 2 minutes. The above mixtures were then added to 18 mM o-phthalaldehyde in 20% ethanol solution. The samples were detected after 1, 5, 10, 20, 30 and 50 minutes respectively using exciting with 436 nm light and detecting with 450˜600 nm. The results are shown in FIG. 3A. In FIG. 3A, the fluorescence of homocysteine-OPA complex rises from 1 minute and achieves the strongest emission at the fifth minute. The fluorescence then decays as the reaction time extends to the fiftieth minute. Further, samples containing homocysteine were added to 100 mM TRIS buffer containing 1 mM EDTA and 100 mM NaBH₄ to react for 2 minutes. The above mixtures were then added to 100 mM o-phthalaldehyde in 20% ethanol solution and the fluorescence were detected at 1, 2, 3, 4, and 5 minutes as shown in FIG. 3B. The fluorescence with 100 mM o-phthalaldehyde addition achieves the highest emission after reacting for one minute and then decays very gradually. Thus, the reaction time for the homocysteine assay according to the invention is preferred at about 4 minutes, that is, the sample is mixed with the competing agent, TRIS buffer, and after 2 minutes o-phthalaldehyde is added to the mixture to form fluorescent complex and the fluorescence is measured 2 minutes after OPA addition.

In a preferred embodiment, 100 mM TRIS containing 1 mM EDTA and 18 mM NaBH₄ is added to a sample containing homocysteine. After 2 minutes, 18 mM o-phthalaldehyde in 20% ethanol solution is added to the sample and the fluorescence of the sample is measured after 2 minutes. The homocysteine concentration is then calculated accordingly.

### EXAMPLE III

A preferred kit and method for homocysteine assay according to one embodiment of the invention is disclosed.

The homocysteine assay includes in this embodiment a competing agent with an amino group (—NH₂). The preferred competing agent is an amine, acetamide, ethylamine or tris-(hydroxymethyl)aminomethane (TRIS). Preferably, the competing agent is 100 mM TRIS buffer containing ethylenediamine tetraacetic acid (EDTA) and sodium borohydride. The kit also comprises a reactive agent that reacts with the amino groups of cysteine, homocysteine and the competing agent. In a preferred embodiment, the reactive agent is an aldehyde, such as 1-100 mM o-phthalaldehyde (OPA) in 20% ethanol or methanol solution. The reactivity of the competing agent and the reactive agent should be higher than cysteine and the reactive agent but lower than homocysteine and the reactive agent. The reactive agent only forms fluorescent compound with homocysteine but not with the competing agent.

Standard Addition

Human Serum samples were spiked with 50, 100 and 150 μM homocysteine. 1 ml serum sample was added to 1 ml 100 mM TRIS buffer, pH 7, containing 1 mM EDTA and 10 mM sodium borohydride. The mixture was incubated for 2 minutes at room temperature, and then combined with 1 ml o-phthaldehyde (100 mM) in 20% ethanol. The fluorescence of the resulting mixture was monitored at 2 minutes after o-phthaldehyde addition by exciting with 436 nm light and the fluorescence is measured maximum at 525 nm. The results are shown in Table 1.

TABLE 1

| Homocysteine | | | | |
|---|---|---|---|---|
| Concentration | Homocysteine Standard | | Serum Addition | |
| (μM) | Fl Intensity | Δ Intensity | Fl Intensity | Δ Intensity |
| 0 | 0.534 | 0.000 | 1.779 | 0.024 |
| 50 | 0.545 | 0.011 | 1.891 | 0.136 |
| 100 | 0.552 | 0.018 | 2.028 | 0.273 |
| 150 | 0.560 | 0.026 | 2.063 | 0.308 |

The intensity of serum blank sample: 1.221
The intensity of blank sample (TRIS buffer and OPA mixture): 0.534

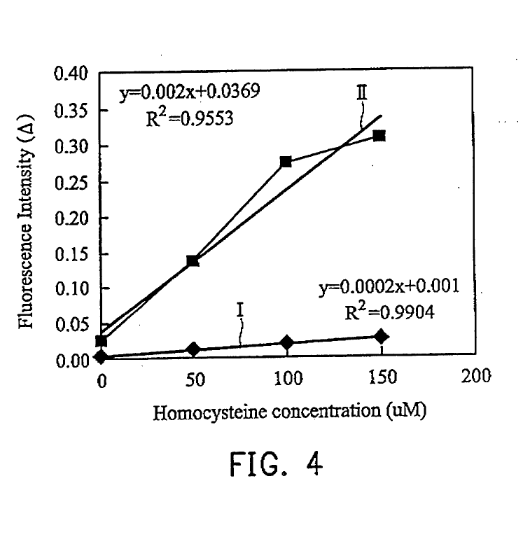

According to Table 1, detection curves are drawn as shown in FIG. 4, wherein curve I is the calibration curve of homocysteine standard: y=0.0002x+0.001, and curve II is the standard addition curve of homocysteine in serum: y=0.002+0.0369.

Sample Detection

1 ml of a bio-sample, e.g. serum or urine, is mixed with 1 ml 100 mM TRIS buffer containing 1 mM EDTA and 18 mM sodium borohydride. After standing for two minutes at room temperature, 1 ml 100 mM o-phthaldehyde in 20% ethanol is then added to the bio-sample. After 2 minutes, the sample is excited with 436 nm light and the fluorescence is measured at 525 nm. The homocysteine concentration of the bio-sample can be calculated with the fluorescence reading in accordance with the detection curve.

For a bio-sample, fluorescence is derived from homocysteine reaction product, reagent interference (competing agent and reactive agent), and sample color interference. The latter two interferences can be eliminated by blank tests when experiments are carried out. Homocysteine standard curve (left column of Table 1) is measured using H₂O as matrix. When homocysteine concentration is 0 μM, the fluorescence is mainly contributed by the reagent, regarded as reagent interference. As for serum addition experiment, human serum (containing unknown homocysteine level) is used as matrix with homocysteine addition. Therefore, homocysteine is added to trace back the initial homocysteine level in the sample. It should be noted that serum color contributes to background interference. As a result, serum blank (without buffer and o-phthaldehyde) can be measured as well to eliminate color interference.

While the invention has been described by way of example and in terms of the preferred embodiments, it is to be understood that the invention is not limited to the disclose embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

1. A method for determining homocysteine in a bio-sample, comprising the following steps:
(a). adding a competing agent with an amino group to the bio-sample;
(b). adding an aldehyde compound to mixture (a) to form an fluorescent complex; and
(c). detecting the fluorescent intensity of mixture (b) to determine the amount of homocysteine in the bio-sample.

2. The method as claimed in claim 1, wherein the competing agent is tris-(hydroxymethyl)aminomethane (TRIS), acetamide, ethylamine or amine.

3. The method as claimed in claim 2, wherein the amine is a long-chained, branched, or cyclic compound with at least one amino-group.

4. The method as claimed in claim 1, wherein the bio-sample is serum or urine.

5. The method as claimed in claim 4, wherein the bio-sample contains homocysteine and cysteine.

6. The method as claimed in claim 5, wherein the aldehyde compound forms fluorescent complex only with homocysteine but not with the competing agent.

7. The method as claimed in claim 1, wherein the aldehyde compound is o-phthalaldehyde with a concentration from 1 to 100 mM.

8. The method as claimed in claim 1, wherein the total operation time from step (a) to step (c) is 4 to 15 minutes.

9. The method as claimed in claim 1, wherein the operation time between step (a) to step (b) is about 2 minutes.

10. The method as claimed in claim 9, wherein the operation time between step (b) and step (c) is about 2 minutes.

11. A method for determining homocysteine in the presence of cysteine in a bio-sample, comprising the following steps:
(a). adding a competing agent containing tris-(hydroxymethyl)aminomethane (TRIS) to the bio-sample;
(b). adding an o-phthalaldehyde into mixture (a) to form a fluorescent complex; and
(c). detecting the fluorescent intensity of mixture (b) to determine homocysteine concentration thereof.

12. The method as claimed in claim 11, wherein the competing agent further comprises ethylenediamine tetraacetic acid (EDTA) and sodium borohydride.

13. The method as claimed in claim 12, wherein the concentration of tris-(hydroxymethyl)aminomethane (TRIS) is about 100 mM, the concentration of ethylenediamine tetraacetic acid (EDTA) is about 1 mM and the concentration of sodium borohydride is about 18 mM.

14. The method as claimed in claim 12, wherein the concentration of o-phthalaldehyde is from 1 to 100 mM in 20% ethanol solution.

15. The method as claimed in claim 14, wherein the operation time from step (a) to step (c) is from 4 to 15 minutes.

16. The method as claimed in claim 15, wherein the time between step (a) to step (b) is about 2 minutes.

17. The method as claimed in claim 16, wherein the time between step (b) to step (c) is about 2 minutes.

18. The method as claimed in claim 11, wherein the bio-sample is serum or urine.

19. A kit for homocysteine detection in a bio-sample containing homocysteine and cysteine, comprising:
a competing agent, comprising at least one compound with an amino group; and
a reactive agent that forms a fluorescent complex with homocysteine but does not form fluorescent complex with the competing agent.

20. The kit as claimed in claim 19, wherein the reactive stability of the competing agent and the reactive agent is higher than that of cysteine and the reactive agent, but lower than that of homocysteine and the reactive agent.

21. The kit as claimed in claim 19, wherein the competing agent is tris-(hydroxymethyl)aminomethane (TRIS), acetamide, ethylamine or amine.

22. The kit as claimed in claim 21, wherein the competing agent further comprises ethylenediamine tetraacetic acid (EDTA) and sodium borohydride.

23. The kit as claimed in claim 22, wherein the concentration of tris-(hydroxymethyl)aminomethane (TRIS) is about 100 mM, the concentration of ethylenediamine tetraacetic acid (EDTA) is about 1 mM and the concentration of sodium borohydride is about 18 mM.

24. The kit as claimed in claim 21, wherein the reactive agent comprises an aldehyde.

25. The kit as claimed in claim 24, wherein the aldehyde is o-phthalaldehyde.

26. The kit as claimed in claim 24, the concentration of o-phthalaldehyde is from 1 to 100 mM in ethanol solution.
